(19) United States
(12) Patent Application Publication
(10) Pub. No.: US 2004/0087533 A1
(43) Pub. Date: May 6, 2004

(54) Antisense compounds, methods and compositions for treating MMP-12 related inflammatory disorders

(76) Inventors: Andreas Dieckmann, Bromma (SE); Robert Lofberg, Djursholm (SE); Oliver Von Stein, Spanga (SE); Petra Von Stein, Spanga (SE); Liam Good, Stockholm (SE)

Correspondence Address: YOUNG & THOMPSON, 745 SOUTH 23RD STREET 2ND FLOOR, ARLINGTON, VA, 22202

(21) Appl. No.: 10/619,906

(22) Filed: Jul. 16, 2003

Related U.S. Application Data
(60) Provisional application No. 60/407,680, filed on Sep. 4, 2002.

(30) Foreign Application Priority Data
Jul. 18, 2002 (SE) 0202253-1

(51) Int. Cl. A61K 48/00; C07H 21/04

(52) U.S. Cl. 514/44; 536/23.2

(57) ABSTRACT

The present invention relates antisense oligonucleotide compounds for use in modulating the function of nucleic acid molecules encoding mammalian MMP-12. More specifically, the invention provides compounds of 8 to 50 nucleobases in length capable of specifically hybridising with nucleic acid molecules encoding MMP-12 and thereby inhibiting the expression of the MMP-12 protein product, as well as pharmaceutical compositions thereof and methods of its use.

### FIELD OF THE INVENTION

The present invention relates to oligonucleotides as antisense compounds. More specifically, the invention provides antisense oligonucleotides that can act as specific inhibitors of metalloproteinase 12 (herein “MMP-12), as well as pharmaceutical compositions thereof and methods for its use.

### BACKGROUND OF THE INVENTION

Metalloproteinases represent a super family of proteinases (enzymes), whose numbers have increased dramatically in recent years. Based on structural and functional considerations, these enzymes have been classified into families and subfamilies (Hooper, 1994). Examples of metalloproteinases include the matrix metalloproteinases (MMPs) such as the collagenases (MMP1, MMP8, MMP13), the gelatinases (MMP2, MMP9), the stromelysins (MMP3, MMP10, MMP 11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases so called due to the presence of a metal ion in their active center, are primarily involved with the degradation of the extracellular matrix components, a process necessary for the growth and remodelling of tissues, etc. under normal physiological conditions.

MMP-12, also known as macrophage elastase or metalloelastase, was first cloned from the mouse by Shapiro et al (1992) and from man in 1993 by the same group (Shapiro et al, 1993). Due to the extensive involvement of metalloproteinases in a whole range of processes, it is perhaps not surprising that metalloproteinases have been associated with the onset and progression of many diseases types and conditions. For example: various inflammatory and allergic diseases such as, inflammation of the joint (especially rheumatoid arthritis, osteoarthritis and gout), inflammation of the gastro-intestinal tract (especially inflammatory bowel disease, ulcerative colitis, gastritis and Crohn's Disease), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing ; demyelinating diseases of the central and peripheral nervous systems (such as multiple sclerosis); Alzheimer's disease; extracellular matrix remodelling observed in cardiovascular diseases such as restenosis and atheroscelerosis; asthma; rhinitis; and chronic obstructive pulmonary diseases (COPD).

Considerable lines of scientific evidence indicate that uncontrolled connective matrix metalloproteinase (MMPs) activity is responsible for much of the disease promoting effects seen with MMPs, and as a consequence the inhibition of these enzymes has inevitably become an attractive target for therapeutic intervention (Matrisian, 1992; Emonard, et al 1990; Docherty, 1990).

The transition of MMPs inhibitors into a clinical setting has not come without its share of related problems. Broad-spectrum inhibition of MMPs in the clinical setting results in musculoskeletal stiffness and pain (Rasmussen and McCann, 1997). These side effects and others associated with broad-spectrum inhibition may be enhanced in chronic administration. Thus, it would be very advantageous to provide selective MMP inhibitors that are capable of preventing the activity of only the particular MMP of interest.

### PRIOR ART

There are studies indicating that MMP-12 is required for the development of cigarette smoke-induced emphysema in mice (Hautamaki, et al 1997). This is further supported by a publication showing that transgenic mice having no functional intergrin alpha v beta 6 developed MMP-12 dependent emphysema (Morris, et al 2003).

Accelerated breakdown of the extracellular matrix of articular cartilage is a key feature in the pathology of both rheumatoid arthritis and osteoarthritis and current evidence suggests that the inappropriate synthesis of MMPs is the key event. Furthermore, a range of MMPs can hydrolyse the membrane-bound precursor of the pro-inflammatory cytokine tumour necrosis factor alpha (TNF-alpha) (Gearing, et al 1994). This cleavage yields mature soluble TNF-a and the inhibitors of MMPs can block production of TNF-a both in vitro and in vivo (Mohler, et al 1994 and McGeehan, et al 1994).

The observation that MMP- 12 is largely macrophage specific supports the findings that MMP-12 is required by macrophages to enable migration of macrophages into sites of inflammation (Shipley et al, 1996). Furthermore, macrophages themselves are instrumental in the maintenance of inflammation, in that they are responsible for the production of many pro-inflammatory cytokines. Thus targeting MMP-12 and thereby reduce macrophage migration could offer a new therapeutic possibility to treat inflammatory conditions.

To this point, there appears to be very few selective inhibitors of MMP-12 reported and no selective or non-selective inhibitor of MMP-12 has been approved or marketed for the treatment of any disease in any mammal. Accordingly, there is a need to find new compounds that are potent and selective MMP inhibitors, and that have an acceptable therapeutic index of toxicity/potency to make them amenable for use clinically in the prevention and treatment of the associated disease states.

The object of this invention is to provide such selective compounds in the form of antisense oligonucleotides.

### SUMMARY OF THE INVENTION

The present invention provides antisense oligonucleotide compounds for use in modulating the function of nucleic acid molecules encoding mammalian MMP-12, ultimately by modulating the amount of MMP-12 produced. More specifically, the invention provides compounds of 8 to 50 nucleobases in length capable of specifically hybridising with nucleic acid molecules encoding MMP-12 and thereby blocking the production of the MMP-12 protein product. Further provided are methods and compositions of modulating the expression of MMP-12 in cells or tissues comprising contacting said cells or tissues with one or more of the antisense compounds or compositions of the invention. This is achieved by providing antisense compounds which specifically hybridise with nucleic acids encoding the MMP-12 protein product.

The invention is further defined in the attached claims, incorporated herein by reference.

### DESCRIPTION OF DRAWINGS

The invention will be described in closer detail in the following description, examples, and attached drawings, in which:

FIG. 1 shows RT-PCR analysis of MMP-12 expression on biopsy samples from patients afflicted with either ulcerative colitis (A) or with Crohn's Disease (B). The experimental protocol is out-lined in example 2. (Key: M is a base-pair marker; H represents a biopsy from a totally normal healthy individual; C represents a biopsy sample taken from a non-inflamed area; and T represents a biopsy taken from an inflamed area from the same patient. Numbers in brackets indicates patient number and the horizontal bar denotes a C and T biopsy sample derived from the same patient). Alpha actin is used as a loading control and indicates the expression status of a house-keeping gene used commonly to demonstrate equal MRNA input in all RT-PCR reactions.

FIG. 2 shows a histogram depicting 4 different criteria used to assess improvement in the degree of inflammation of the gastrointestinal tract after administration of an antisense compound. In this example, the antisense compound is that given by SEQ.ID.NO 3 and the experimental protocol is given by example 2. (Key: The black solid bar denotes healthy animals that received only standard drinking water (healthy control). The hatched bar denotes colitis induced animals who receive 2.5% DSS in their drinking water which will induce inflammation of the colon (sick control). The chequered bar denotes those animals who received in addition to DSS in their drinking water, antisense compound SEQ.ID.NO 3 as outlined in example 7). Thick black bars denote negative control vs. sick animal control. Thin black bars denote comparison of sick animal control vs. MMP-12 antisense treated group. Histology was graded 0-4 according to the scale shown in Table 1. Significance is indicated as * P<0.05, ** P<0.001 and *** P<0.0005. Error bars: SEM.

FIG. 3 shows histological sections of mouse colonic tissue as given by example 2. A) Healthy colon with normal border epithelium, normal crypts of Lierberkiihn and few inflammatory cells, B) Inflamed colon showing depletion of border epithelium, disturbed crypt architecture and a massive infiltration of inflammatory cells, C) MMP-12 antisense as given by SEQ ID NO. 3 treated colon showing amelioration of inflammation with disturbed but conserved epithelial border, normalized crypts and little inflammatory cell infiltration. On top of the epithelial border, a layer of mucus can be seen. Bars=50 μm.

### DETAILED DESCRIPTION OF THE INVENTION

Before the present method is disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

In the context of the invention, “antisense” as in “antisense molecules” and “antisense sequences” refers to single stranded RNA or DNA molecules complementary to a portion of the mRNA of a target gene. The antisense molecule will base-pair with the MRNA, thus preventing translation of the mRNA into protein. Consequently, the term “antisense therapy” refers to methods using such antisense compounds which specifically hybridise to a target nucleic acid and modulate its function or translation, for example by suppressing or reducing the expression of gene products coded by said sequence.

In the context of the present invention, “complementary” refers to the capacity for precise pairing between two nucleotides.

Within the context of the present invention, “hybridization” refers to hydrogen bonding, which may be Watson-Crick, Hoogsteen or reverse Hoogsteen hydrogen bonding, between complementary nucleoside or nucleotide bases. Thus complementarity and hybridisation are terms used to indicate a sufficient degree of complementarity or precise paring such that stable and specific binding occurs between the oligonucleotide and the DNA or RNA target.

An antisense compound is specifically hybridisable when binding of the compound to the target DNA or RNA molecule interferes with the normal function of the target DNA or RNA to cause a loss of utility, and there is a sufficient degree of complementarity to avoid non-specific. binding of the antisense compound to non-specific target sequences under conditions in which specific binding is desired.

Furthermore, in the context of the present invention, “hybridisation under stringent conditions” refers to the criteria regarding temperature and buffers well know to those skilled in the art (Ausubel et al., 1991).

As is well known in the art, “functionally homologous” means sequences sharing perhaps a lower structural homology with the disclosed sequence, but exhibiting homologous function in vivo, in either the healthy or the diseased organism, e.g. coding the same or highly similar proteins with similar cellular functions.

As is well known in the art, “functionally inserted” or “operationally inserted” denotes that a sequence has been inserted in a host genome in such orientation, location and with such promoters and/or enhancers, where applicable, that the correct expression of said sequence occurs.

In the context of the present invention, “modulation” means either an increase (stimulation) or a decrease (inhibition) in the expression of a gene. In the context of the present invention, inhibition is the preferred form of modulation of gene expression and mRNA is the preferred target.

The present invention provides oligonucleotide compounds for use in modulating the function of nucleic acid molecules encoding a mammalian MMP-12, ultimately by modulating the amount of MMP-12 produced. More specifically, said compound is an antisense oligonucleotide complementary to the mRNA of the MMP-12. The modulation is achieved by providing antisense compounds, which specifically hybridise with nucleic acids encoding the MMP-12 protein product and thereby inhibit the translation of the MMP-12. In one embodiment the target sequence is human and the antisense compound preferably hybridises to SEQ ID NO. 1 (GenBank® Acc. No. NM-002426) or equivalent functional homologues thereof.

In another embodiment the target sequence is the murine sequence of SEQ ID NO. 2 (GenBank® Acc. No. M82831 )or equivalent functional homologues thereof.

The antisense compounds directed towards one or the other of the above target sequences, or equivalents thereof, in accordance with this invention preferably comprise from about 8 to about 50 nucleobases in length. Antisense oligonucleotides comprising from about 8 to about 30 nucleobases (i.e. from about 8 to about 30 linked nucleosides in length) are particularly preferred, and oligonucleotides comprising about 16 to about 24 nucleobases are most preferred.

The antisense oligonucleotide according to the invention is either a DNA molecule or a RNA molecule. The invention makes available nucleic acid molecules in the form of antisense oligonucleotide molecules as defined above, and in particular SEQ ID NO. 3-14 (See Table 2, and attached Sequence Listing, prepared using Patentln 3.1), capable of specifically hybridising with nucleic acid molecules encoding MMP-12 and thereby blocking the production of the MMP-12 protein product.

In yet a further context of this invention, the term “oligonucleotide” refers to an oligomer or polymer of ribonucleic acid (RNA) or deoxyribonucleic acid (DNA) or mimetics thereof. This term also covers those oligonucleotides composed of naturally-occurring nucleobases, sugars and covalent internucleoside (backbone) linkages as well as oligonucleotides having non-naturally-occurring modifications. As is known in the art, the phosphate groups within the oligonucleotide structure are commonly referred to as forming the intemucleoside backbone of the oligonucleotide. The natural linkage or backbone of RNA and DNA is a 3′ to 5′ phosphodiester linkage.

Specific examples of preferred antisense compounds useful in this invention include oligonucleotides containing modified backbones or non-natural internucleoside linkages. These modifications have allowed one to introduce certain desirable properties that are not offered through naturally occurring oligonucleotides, such as reduced toxic properties, increased stability against nuclease degradation and enhanced cellular up-take.

According to a further embodiment, said antisense oligonucleotide comprises at least one modified nucleobase, which may be chemically modified by substitution in a non-bridging oxygen atom of the antisense nucleic acid backbone with a moiety selected from the group consisting of methane phosphate, methyl phosphate and phosphorothioate. phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates and thionoalkylphosphotriesters.

According to one embodiment, said substitution may take place at one or more nucleotides independently selected from the final three nucleotides at the 3′ terminus and/or 5′ terminus of said oligonucleotide. It is also conceived, that the substitution can occur at any position along the entire length of said oligonucleotide, or indeed all intranucleoside linkages are subjected to modification. Preferably, said oligonucleotide comprises at least one modified sugar moiety nucleobase, and the modified sugar moiety may be a 2′-O-methoxyethyl sugar moiety.

Said antisense agent may also be an antisense agent composed of DNA or RNA or an analogue or mimic of DNA or RNA including but not restricted to the following: methylphosphonate, N3′->P5′-phosphoramidate, morpholino, peptide nucleic acid (PNA), locked nucleic acid (LNA), arabinosyl nucleic acid (ANA), fluoro-arabinosyl nucleic acid (FANA) methoxy-ethyl nucleic acid (MOE). Preferably said antisense agent is a homo or heteropolymer containing combinations of the above DNA or RNA or analogues or mimics of DNA or RNA.

In a further embodiment, the antisense compounds of the present invention can be utilized for therapeutics and as prophylaxis. For therapeutics, an animal, preferably a human, suspected of having a disease or disorder associated with inappropriate expression of MMP-12 which can be treated by modulating the expression of MMP-12, is treated by administering a therapeutically or prophylactically effective amount of antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically (i.e., to delay the onset of a disease or condition in which MMP-12 is suspected of being involved).

In yet another embodiment, the antisense compounds of the invention are useful for research and diagnostics of human subjects, because these compounds hybridize to nucleic acids encoding MMP-12, enabling sandwich and other assays to easily be designed to exploit this fact. Hybridization of the antisense oligonucleotides of the invention with a nucleic acid encoding MMP-12 and the resulting suppression/inhibition in expression of MMP-12 can be detected by means well known in the art. For example, radiolabelling of the antisense compound, RNase protection assays, will demonstrate specific hybridisation of the antisense compound to the target MRNA of MMP-12. Various means of detecting reduced levels of MMP-12 can be employed well known in the art such as antibody detection of MMP-12, or enzymatic based activity assays. In another embodiment the antisense compounds are used in a method of inhibition of the expression of MMP-12 in cells or tissues, wherein said cells or tissues are contacted in vivo or in vitro with a therapeutically effective dose of the compound or composition of the invention, thereby inhibiting the expression of MMP-12. Preferably, said inhibition suppresses a MMP-12 dependent process in a human subject. The MMP-12 dependent process is most preferably one of inflammatory bowel disease, such as ulcerative colitis and Crohn's disease, rheumatoid arthritis, psoriasis, emphysema and asthma.

Another embodiment of the invention relates to a method of diagnosing inflammatory bowel disease in a human subject comprising screening for the presence or absence of the expression of MMP-12 and the expression of MMP-12 is an indication of inflammatory bowel disease.

Because they are selective, the compounds of the present application are expected to be useful for long-term therapy with less of the complications related to known broad spectrum inhibition. Thus, while the compounds of the present application are useful for the treatment of a variety of MMP-12 mediated diseases and conditions, these selective inhibitors are particularly useful for the treatment of disorders that have a significant inflammatory component.

As an alternative to targeted antisense delivery, targeted ribozymes may be used. The term “ribozyme” refers to an RNA-based enzyme capable of targeting and cleaving particular base sequences in both DNA and RNA. Ribozymes can either be targeted directly to cells, in the form of RNA oligonucleotides incorporating ribozyme sequences, or introduced into the cell as an expression vector encoding the desired ribozymal RNA. Ribozymes may be used and applied in much the same way as described for antisense polynucleotide. Ribozyme sequences also may be modified in much the same way as described for antisense polynucleotide. For example, one could incorporate non-Watson-Crick bases, or make mixed RNA/DNA oligonucleotides, or modify the phosphodiester backbone.

Another alternative to antisense is the use of so called “RNA interference” (RNAi). Double-stranded RNAs (dsRNAs) can provoke gene silencing in numerous in vivo contexts including Drosophila, Caenorhabditis elegans, planaria, hydra, trypanosomes, fungi plants and mammals. The natural function of RNAi and co-suppression appears to be protection of the genome against invasion by mobile genetic elements such as retrotransposons and viruses which produce aberrant RNA or dsRNA in the host cell when they become active (Jensen et al., 1999; Ketting et al., 1999; Ratcliff et al., 1999; Tabara et al., 1999). Specific mRNA degradation prevents transposon and virus replication although some viruses are able to overcome or prevent this process by expressing proteins that suppress PTGS (Lucy et al., 2000). The double-stranded RNA molecule may be prepared by a method comprising the steps: (a) synthesizing two RNA strands each having a length from 19-25, e. g. from 19-23 nucleotides, wherein said RNA strands are capable of forming a double-stranded RNA molecule, wherein preferably at least one strand has a 3′-overhang from 1-5 nucleotides, (b) combining the synthesized RNA strands under conditions, wherein a double-stranded RNA molecule is formed, which is capable of mediating target-specific nucleic acid modifications, particularly RNA interference and/or DNA methylation. In one embodiment the antisense RNAi comprises at least an 8 nucleotide portion included in one of the sequences of SEQ.ID.NO 3-14, and has a total length of no more than 25 nucleotides.

The dsRNA is usually administered as a pharmaceutical composition. The administration may be carried out by known methods, wherein a nucleic acid is introduced into a desired target cell in vitro or in vivo. Commonly used gene transfer techniques include calcium phosphate, DEAE-dextran, electroporation, microinjection and viral methods. Such methods are taught in Current Protocols in Molecular Biology, Ausubel et al., (1993).

The present invention also makes available a pharmaceutical composition, wherein said composition comprises a compound or antisense agent as describe above, and a pharmaceutically acceptable formulation and composition, carrier- or diluent. Said pharmaceutical composition preferably further comprises a colloidal dispersion system. The pharmaceutical composition of the present invention may be administered in a number of ways depending largely on whether a local, topical or systemic mode of administration is most appropriate for the condition to be treated. These different modes of administration are for example topical (e.g., on the skin), local (including ophthalmic and to various mucous membranes such for example vaginal, nasal, and rectal delivery), oral or parenteral and pulmonary.

The preparation of such compositions and formulations is generally known to those skilled in the pharmaceutical and formulation arts and may be applied to the formulation of the composition of the present invention.

In the scope of this invention, preferred examples of pharmaceutically acceptable salts include but are not limited to (a) salts formed with cations such as sodium, potassium, ammonium, magnesium, calcium, polyamines such as spermine and spermidine, etc. (b) acid addition salts formed with inorganic acids, for example hydrochloric acid, hydrobromic acid, nitric acid, phosphoric acid, sulfuric acid and the like; (c) salts formed with organic acids such as, for example, acetic acid, alginic acid, ascorbic acid, benzoic acid, citric acid, fumaric acid, gluconic acid, maleic acid, methanesulfonic acid, naphthalenedisulfonic acid, naphthalenesulfonic acid, oxalic acid, palmitic acid, polyglutamic acid, p-toluenesulfonic acid, polygalacturonic acid, succinic acid, tartaric acid, tannic acid and the like; and (d) salts formed from elemental anions such as chlorine, bromine, and iodine.

In yet another embodiment, pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavouring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Generally, such carriers should be non-toxic to the recipient at the dosages and concentrations used. Ordinarily, the preparation of such compositions involves combining the therapeutic agent with one or more of the following: buffers, antioxidants, low molecular weight polypeptides, proteins, amino acids, carbohydrates including glucose, sucrose or dextrins, chelating agents such as EDTA, glutathione and other stabilizers and excipients. Neutral buffered saline or saline mixed with non-specific serum albumin are examples of suitable diluents.

The pharmaceutical formulations of the present invention, which may conveniently be presented in unit dosage form, may be prepared according to conventional techniques well known in the pharmaceutical industry.

In yet another embodiment, the compositions of the present invention may be prepared and formulated as emulsions which are typically heterogeneous systems of one liquid dispersed in another in the form of droplets (Idson, 1988). Examples of naturally occurring emulsifiers used in emulsion formulations include acacia, beeswax, lanolin, lecithin and phosphatides. The application of emulsion formulations via dermatological, oral and parenteral routes and methods for their manufacture have been reviewed in the literature (Idson, 1988).

In one embodiment of the present invention, the compositions of oligonucleotides and nucleic acids can be formulated as microemulsions. A microemulsion is defined as a system of water, oil and amphiphile, which is a single optically isotropic and thermodynamically stable liquid solution (Rosoff, 1988). Another embodiment of the present invention is the use of liposomes for the transfer and delivery of active ingredients to the site of action. Because the liposomal membrane is structurally similar to biological membranes, when liposomes are applied to a tissue, the liposomes start to merge with the cellular membranes. This fact has prompted extensive research in the use of liposomes as potential drug delivery modes.

In another embodiment, the use of penetration enhancers may be of use as a mode of drug delivery. Such agents are classified as belonging to one of five broad categories, i.e., surfactants, fatty acids, bile salts, chelating agents, and non-chelating non-surfactants (Lee et al., 1991).

In another related embodiment, compositions of the invention may contain one or more antisense compounds, particularly oligonucleotides, targeted to a first nucleic acid and one or more additional antisense compounds targeted to a second nucleic acid target. Two or more combined compounds may be used together or sequentially.

The formulation of therapeutic compositions and their subsequent administration is believed to be within the skill of those in the art. Optimal dosing schedules can be calculated from measurements of drug accumulation in the body of the patient. Persons of ordinary skill can easily determine optimum dosages, dosing methodologies and repetition rates. Following successful treatment, it may be desirable to have the patient undergo maintenance therapy to prevent the recurrence of the disease state, wherein the oligonucleotide is administered in maintenance doses.

The present invention also relates to a recombinant nucleotide sequence comprising an antisense compound according to the invention. The recombinant nucleotide sequence can be inserted in an expression vector, such as a plasmid or virus or any other vector known to a person skilled in the art. Thus, the invention includes the antisense oligonucleotide sequences operably linked to one or more expression control elements, such that in vivo or in vitro expression of said antisense compound could be achieved. The vector capable of harbouring said antisense oligonucleotides can be of eukaryotic or prokaryotic origin.

One embodiment of the invention is a method of inhibiting the expression of MMP-12 in cells or tissues, wherein said cells or tissues is contacted in vivo or in vitro with the recombinant nucleotide sequence expressed by the recombinant vector. The invention also includes host cells transformed with these antisense oligonucleotide sequences operably linked to one or more expression control elements.

The invention in particular provides compounds and methods for the treatment of an animal, particular a human suspected of having or being prone to a human disease associated with inappropriate modulation of MMP-12, by administrating a therapeutic or prophylactically effective amount of one or more antisense compound or compositions of the invention designed to modulate expression of MMP-12.

The present invention also provides transgenic cells as such, as well as transgenic non-human animals. Transgenic animals include animals comprising viable transgenic cells, or transgenic organs, as well as entire animals functionally incorporating any one of the inventive antisense oligonucleotide sequences (SEQ ID NOs. 3 to 14) or functional parts thereof, in their genome under the control of a suitable expression cassette. Such animals are useful as research tools for investigations regarding the aetiology of MMP- 12 related disorders, the progression, diagnosis and treatment of the same. As is well know in the art, these expression cassettes containing suitable promoters and enhancers are introduced into the cell of interest in the form of vectors such that expression of the desired antisense DNA sequence is achieved, resulting in an in vitro or in vivo inhibition of the production of MMP-12. Thus, in one embodiment the inventive sequences may be over-expressed, such that suppression of the intended target is achieved in the cells in which the antisense compound is expressed.

One embodiment of the present invention is thus such transgenic cells, organs or animals, and their use as models for investigating the nature and/or aetiology of MMP-12 related diseases, as models for evaluating the efficacy of pharmaceuticals against such diseases, as well as investigating the effect of known and suspected causative agents behind such diseases.

Having confirmed the involvement of MMP-12 in inflammatory disorders, the invention further provides screening assays for identifying an agent that modulates the activity of MMP-12 by altering the activation of the MMP-12 molecule

As the agent usable in the screening method of the invention, a newly synthesized compound, a commercial compound or a known compound which is registered in a chemical file but the various activities are unknown, a series of compounds obtained by the technology of combinatorial chemistry can be used. Also, a supernatant of culture of a microorganism, a natural component derived from a plant or a marine organism, an animal tissue extract, and the like can be used.

The method comprises contacting the MMP-12, under conditions that allow an agent suspected of being able to alter the activity of MMP-12 to interact with MMP-12 such that a change in activity levels of MMP-12 can be easily seen. A preferred mode of changes in activity levels is the inhibition of MMP-12 activity or an agent with antagonistic effect.

According to the present invention, an assay for identifying an agent that alters the specific activity of MMP-12 can be, for example, an in vitro, or cell based assay and a preferred manner of monitory changes in MMP-12 activity could be for example measuring MMP-12 proteolytic activity against some of the know substrates of MMP-12 such as tropoelastin, osteonectin, vitronectin, and fibronectin in the form of a zymography assay. Such methods are taught in for example Ausubel et al., 1991.

In another embodiment, screening of compounds that have an antagonistic effect of MMP-12 can be done using solid phase combinatorial library approach.

The library can be, for example, be constructed as a one-bead-two-compounds library so that every bead contained a common quenched fluorogenic substrate and a different putative inhibitor. After incubation with MMP-12, beads containing active inhibitors can be simply collected and the inhibitor compound structure analyzed using, for example a MALDI-TOF mass spectrometer (Franz et al., 2003).

While the present invention has been described with specificity in accordance with certain of its preferred embodiments, the following examples serve only to illustrate the invention and are not intended to limit the same. While they are typical of those that might be used, other procedures known to those skilled in the art may alternatively be adopted without resort to undue experimentation.

### EXAMPLES

### Example 1

### Identification of MMP-12 as being over-expressed in human conditions of inflammation

Collection of the Appropriate Biopsy Material

The biopsies were taken from patients who were selected on the basis of. clinical and pathological evidence of having the inflammatory condition of CD or UC. A total of three biopsies were collected from an inflamed site in the colon, together with three biopsy samples from a non-inflamed region of a single individual patient. This was done for a total of 16 different patients of which eight were diagnosed for CD (patient 1-8) and eight for UC (patient 9-16). The UC patient group comprised 2 females and 6 males, the age range being 29-77 years. The CD age group correspondingly 3 females and 5 males, age range 27-59

The biopsies of each anatomical site of one patient were pooled and total RNA was isolated using Quiagen Rneasy Kit and a Pellet Pestel Motor Homogenizer according to the manufacturers protocol. 32 samples of total RNA were isolated, two samples per patient: inflamed (target) and non-inflamed (control).

Performing cDNA Synthesis of the RNA

Two microgram of each RNA sample was used for a first strand cDNA synthesis using 10pM of the Oligo-dT-primer dT-joint (5′-TAG TCT ATG ATC GTC GAC GGC TGA TGA AGC GGC CGC TGG AGT TTT TTT TTT TTT TTT TTV-3′ (SEQ. ID. NO. 15.) introducing to every synthesised cDNA molecule three restriction enzyme cutting sites: SalI, NotI and BpmI. The buffer, deoxynucleotide triphosphates (dATP, dCTP, dGTP and dTTP) and the enzyme reverse transcriptase (Superscript II) were purchased from Gibco BRL and the reactions were performed according to the guidelines of the manufacture. The reaction mixture for the first strand synthesis excluding the enzyme was preincubated for 5 min at 65° C. in a PCR machine (PCR sprint from Hybaid), chilled on ice, and then preheated to 42° C., before the enzyme Superscript II was added and the mixture incubated for 1 h at 42° C. in a PCR machine (PCR sprint from Hybaid).

For the second strand synthesis, 41 ul second strand buffer mix were added to the reactions according to the provided protocol (Gibco BRL) and 4μl E.coli Polymerase I (New England Biolabs), 1.5 μl E.coli DNA ligase (New England Biolabs) and 0.7 μl Rnase H (Gibco BRL) in a total volume of 160 μl. The reactions were incubated for 2.5 h at 16° C. in the PCR machine PCRsprint and then purified using the Quiagen PCR Purification Kit according to the protocol provided. The samples were eluted with 32 μl of elution buffer and 26 μl of each sample was used for the following steps.

Amplification of the 3 ′-termini of the cDNAs

Due to limited amounts of material obtained from the biopsies, a pre-amplification step was necessary. For in vitro amplification of the 3′-end of cDNAs, 26 μl of cDNA from all samples were digested with 10U of the restriction enzyme DpnII in a volume of 30 μl for 3 h at 37° C. The cut cDNAs were purified once more using Quiagen PCR purification Kit and the cDNAs were eluted in 47 μl elution buffer. The following circular ligation step was performed in a volume of 50 μl including 44 μl of the DpnII cut cDNA and 2000U T4 DNA ligase (New England Biolabs). These reaction mixtures were incubated at 22° C. for 1 h, heat inactivated by 65° C. for 10 min and 25 μl of each reaction mixture was used for the amplification step. A mixture for 5 PCR-reactions per sample was prepared (5×50 μl=250 μl in total) containing 25 μl cDNA (DpnII cut and circular ligated), 25 μl 10× Advantage 2 PCR buffer (Clontech), 5 μl joint-Not primer (10 pmol/μl; 5′-TGA TGA AGC GGC CGC TGG-3′ (SEQ. ID. NO. 16.)), 5 μl joint-Sal primer (10pmol/μl; 5′-TTC ATC AGC CGT CGA CGA TC-3′ (SEQ. ID. NO. 17.), 5μl 10mM dNTP mix and 5 μl 50×Advantage 2 Taq-Polymerase (Clontech). For each sample the PCR mixture was distributed into 5 PCR reaction tubes and PCR performed under the following conditions: 1 min 94° C. then 16×(20 sec 94° C., 20 sec 55° C., 1 min 72° C.).

Four reactions per sample were removed and placed on ice and the optimal cycle number was determined with one of the reactions per sample. The optimal cycle number was determined to 18 cycles for all 32 samples, thus for the remaining four reactions per sample two additional cycles [2×(20 sec 94° C., 20 sec 55° C., 1 min 72° C.)] were performed. The four PCR reactions per sample were pooled (to total volume of 200 μl) and subsequently purified using the Quiagen PCR purification Kit in which the DNA was eluted with 34 μl elution buffer. The purified reactions were the starting material for the identification of the differentially expressed genes protocol.

Isolation of the Differentially Expressed cDNA (Subtraction Protocol) from Human Biopsies

Isolation of differentially expressed cDNAs was performed according to the protocol outlined in (von Stein OD, 2001) with minor modifications to the protocol.

Screening for the Differentially Expressed Genes

For construction of a cDNA library, 2 000 clones were plated out from each subtraction on one 22 cm² agar plate. From these plates 384 colonies were picked and placed in 384 well plates with 70 μl LB medium/well (see Maniatis et al., 1989) (+ampicillin 100 mg/ml) using BioPick machine of BioRobotics (Cambridge, UK). The bacterial clones were incubated over night at 37° C. and then used for colony PCR. This PCR was performed in 384 PCR well plates in a volume of 20 μl per sample. One PCR reaction included: 2 μl 10×PCR buffer, 0.4 μl Sport-Not primer (10 pmol 5′-CGT AAG CTT GGA TCC TCT AGA GC-3′ (SEQ. ID. NO. 18)), 0.4 μl of Sport-Sal primer (10 pmol 5′-TGC AGG TAC CGG TCC GGA ATT CC-3′ (SEQ. ID. NO. 19)), 1.6 μl dNTP mix (25 mM of each nucleotide), 0.4 μl 0.1% Bromphenol blue and 0.5 μl DynAzyme Taq-polymerase (2 U/μl; Finnzyme). A master mix for all reactions was prepared, distributed and then inoculated with a 384 plastic replica. The PCR cycling parameters were: 2 min 94° C., 37 times (30 sec 94° C.; 30 sec 50° C., 1 min 72° C.) and 5 min 72° C.

Following amplification, PCR reactions were spotted on Hybond N⁺ membrane (Amersham) using Microgrid TAS of BioRobotics. All clones were spotted in duplicate and genomic DNA was used as guide dots. On one filter 383 genes of all four subtractions were positioned. 24 duplicates were made for analysis by hybridisation with different radioactive cDNA probes. These filters were then hybridised with the radioactively labeled subtracted cDNAs of all eight patients. Sixteen filters were used in 16 different hybridisation experiments. For the labelling with Klenow polymerase, 1 μl of the cDNAs was used. The hybridisation protocol was that of Church-protocol as outlined in (Church and Gilbert 1984).

Phospho-imager Fujifilm BAS 1800II with BAS 1800 III R program, Array vision version 6.0 (Imaging Research Inc), sequencing, and BLAST analysis were used to determine the degree of differentially expression and the identity of the isolated differentially expressed genes.

Confirmation of True Differential Expression

To confirm results of the expression profiling experiment, RT-PCR analysis was performed using gene-specific primers and primers for alpha-actin (control). The original cDNAs derived from eight individual UC patients and eight individual CD patients were used. The cDNAs were then diluted 1:250 in distilled water and a 5 μl aliquot used for one single PCR reaction. Reactions were performed in a total volume of 50μl and included 1× PCR buffer (provided with the Taq-polymerase; Finnzyme), 0.5 μl 25 mM dNTP-mix, 10 pM forward and reverse primer of MMP-12 or alpha-actin and 1 unit of DynZyme (Taq-polymerase of Finnzyme). PCR reactions were performed in Thermohybaid Thermocycler under the following conditions: 1 min 94° C. and N cycles (30sec 94° C., 30sec 55° C., 1 min 72° C.) and 5 min 72° C. For MMP-12 30 cycles were performed (N=30), for α-actin 28 cycles (N=28). Upon completion, 5 μl of each reaction was loaded on a 1×TAE agarose gel and later stained with ethidium bromide.

| | | |
|---|---|---|
| MMP-12 forward: | 5′-GAC TTC CTA CTC CAA CGT ATC ACC -3′ | (SEQ.ID.NO 20) |
| MMP-12 reverse: | 5′-CTC AGT CCA AGG ATG TTA GGA AGC -3′ | (SEQ.ID.NO 21) |
| alpha-actin.forward: | 5′-GTG CAG GGT ATT AAC GTG TCA GGG-3′ | (SEQ.ID.NO.22) |
| alpha-actin.reverse: | 5′-CCA ACT CAA AGC AAG TAA CAG CCC ACG G-3′ | (SEQ.ID.NO.23) |

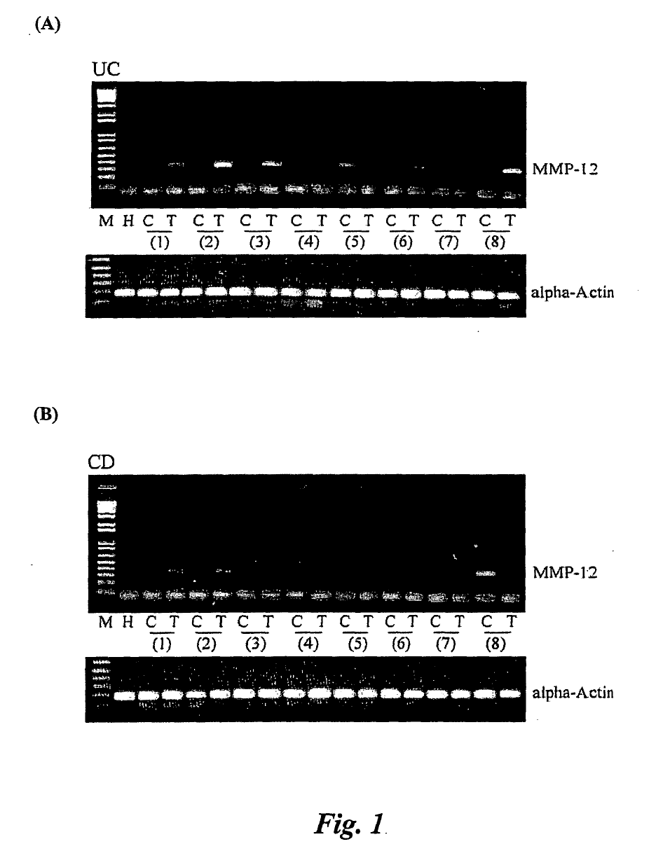

From these analyses it can be concluded that in both conditions of human UC and CD, there is an up-regulation of MMP-12 in the majority of cases (See FIG. 1).

### Example 2

### Analysis of Antisense Oligonucleotide Inhibition of MMP-12

Antisense modulation of MMP-12 expression can be assayed in a variety of ways well known in the art. For example, mRNA levels of MMP-12 can be quantified by, e.g., Northern blot analysis, competitive polymerase chain reaction (PCR), or real-time PCR (RT-PCR). RNA analysis can be performed on total cellular RNA or poly (A)+mRNA.

Methods of RNA isolation are described in, for example, Ausubel, et al., 1992. Northern blot analysis is routine in the art and is described in, for example, Ausubel, et al., 1992. Real-time quantitative PCR can be conveniently accomplished using the ABI PRISM.TM. 7700 Sequence Detection System, available from PE-Applied Biosystems, Foster City, Calif., USA and used according to manufacturer's instructions. Other methods of PCR are also known in the art.

Like wise, MMP-12 protein levels can be quantified in a variety of ways well known in the art, such as immunoprecipitation, Western blot analysis (immunoblotting), or ELISA. Antibodies directed to MMP-12 can be commercially acquired or an antibody can be generated via conventional antibody generation methods. Methods for preparation of polyclonal antisera are taught in, for example, Ausubel, et al., 1997. Preparation of monoclonal antibodies is taught in, for example, Ausubel et al., 1997.

Immunoprecipitation methods well known in the art can be found in, for example, Ausubel, et al., 1998. Western blot (immunoblot) analysis is standard in the art and is described in, for example, Ausubel, et al., 1997. Enzyme-linked immunosorbent assays (ELISA) are standard in the art and are described in, for example, Ausubel, et al., 1991.

Suppression of Inflammation in a Colitis Mouse Model using Antisense Oligonucleotides to MMP-12

An animal model wherein inflammation in the large intestine of mice is induced has been described by Okayasu et al., 1990. In the model used in the present experiment, oral dextran sulfate sodium (DSS) is utilized to induce inflammation (Axelsson, et al., 1998). DSS can be given to the mice in the drinking water, thereby inducing a colitis resembling inflammatory bowel disease (IBD) in man. An MW of about 40-50 kD and an high content of up to about 19% sulphur has been shown to be optimal for the inflammation inducing form of DSS. In Okayasu, 1990, the DSS was given to the animals at a concentration of about 2-5%.

In this study DSS was used at a concentration of 2.5%, dissolved in water, with a final pH of 8.5 (adjusted with NaOH). DSS was given orally to female SPF NMRI mice for 8 consecutive days to induce a stable colitis in all individuals. This type of experimentally induced colitis has been shown to be fully induced at day 4-5 after addition in the drinking water (Cooper et al., 1993).

The antisense substance, as given by SEQ. ID.NO.3, was administered rectally to non-medicated or anaesthetized colitic animals. A shortened XRO feeding tube (Vygon, Ecouen, France) was inserted rectally, up to the level of the ligament of Treitz, and the substance, in a volume of 100 μl, was administered during slow careful retraction of the tubing to avoid rectal leakage of the substance. A single dose of 100 μg antisense in 100 μl water was administered. Therapeutic treatment was given once on day 8 while the DSS treatment continued another 10 days. On day 18 the animals were killed and subjected to analysis of clinical inflammatory parameters and histopathological examinations.

Clinical Signs

Each mouse was observed once daily during the study period. All signs of bad health and any behavioural changes were recorded. Animals showing severe signs of disease and losing more than 15% of its original body weight were killed.

Mortality and Necropsy

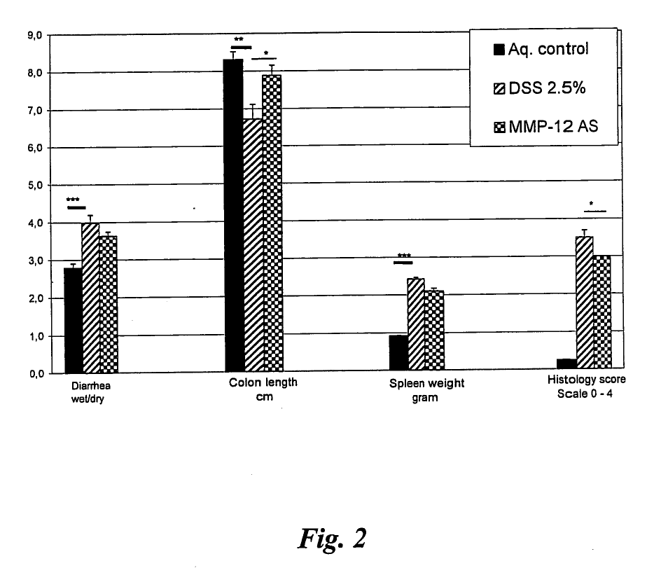

Mortality during the experimental period was recorded. At the end of the experimental period, animals were killed by dislocation of the cervical spine. The abdomen was opened and the spleen was resected and weighed. The large intestine was excised from the ileocecal junction to the proximal rectum, close to its passage under the pelvisternum. The caecum was opened at the apex and feces were carefully removed. The colon was opened longitudinally and the faeces were carefully removed with a spatula. Evaluation of colitis was made by recording clinical parameters such as mortality, colon length, spleen weight and diarrhoea, calculated as wet/dry weight of the faeces after drying 48 h at 60° C. (FIG. 2). The entire caecum and colon were fixed in 4% neutral buffered formaldehyde for microscopic examination.

It is evident from FIG. 2 that there is a specific and significant improvement in all measured parameters. That is to say, treated animals had less diarrhoea, had a more normal colon length, a more normal spleen weight and showed statistically significant signs of histological improvement.

Processing and Microscopic Examination

After fixation, the tissues sampled for microscopic examination were trimmed and specimens were taken from caecum and the mid portion of colon for histological processing. Additional specimens were taken when the first sample was difficult to interpret. The specimens were embedded in paraffin and cut at a nominal thickness of 5 μm, stained with haematoxylin and eosin, and examined under light microscope.

Verification of colitis and estimation of inflammation was performed by an experienced veterinary pathologist, having extensive experience of the histopathological evaluation of DSS-induced colitis in mice. Diagnostic histopathology is based on a standardized grading system shown in Table 1.

TABLE 1
Histopathologic grading system
Colitis lesions:

| | |
|---|---|
| +/− | very mild (may be normal) (0) |
| + | mild (1) |
| ++ | moderate (2) |
| +++ | severe (3) |
| ++++ | very severe (4) |

Histological Analysis of Colonic Sections

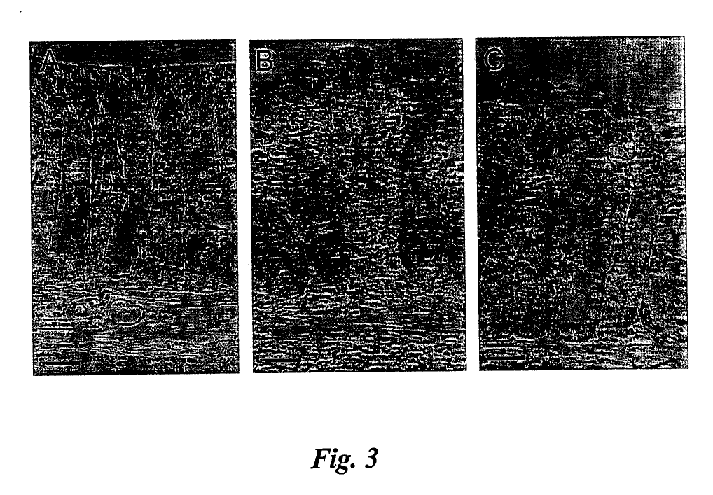

As outlined above, sections taken from the caecum and the mid portion of the colon were used for histological processing. Staining was performed with haematoxylin and eosin. Sections were then examined by light microscopy and morphological changes noted. From FIG. 3 it can be concluded that a single rectal administration of antisense compound as given by SEQ.ID.NO 3, was sufficient to dramatically reduce the inflammation as seen on both physiological parameters and histology (FIGS. 2 and 3).

### Example 3

### In vitro Screening of Human MMP-12 mRNA for Binding Site Accessible to Antisense Sequences

The effect of antisense compounds on target nucleic acid expression can be readily monitored in a variety of cell types provided that the target nucleic acid is present at measurable levels. To those skilled in the art, there are a number of well established methods which can be employed to determine changes in levels of expressed target (See below).

Treatment with Antisense Compounds

In order to identify an antisense compound that exhibits selective binding to human MMP-12 MRNA, and as a consequence causes a reduction in the amount of MMP-12 protein, a proprietary in vitro screening systems was set up by the inventors. Within the coding region of SEQ. ID. NO. 1 (GenBank®( accession no. NM-002426), the inventors surprisingly identified a number of antisense sequences exhibiting very high inhibition.

The antisense sequences where then monitored for their ability to reduce the amount of human MMP-12 mRNA, by use of methods well known in the art, for example, PCR or Northern blot analysis to monitor the levels of target MRNA, whereas western blots indicate levels of protein encoded by the target mRNA. The potency of the antisense sequences was arbitrarily scored as a measure of degree of inhibition of the target sequence. Table 2 lists the antisense sequences in groups in decreasing order of their potency.

TABLE 2

The antisense seQuences grouped in decreasing
order of potency

| Sequence | |
|---|---|
| identification no. | Antisense sequences |
| SEQ.ID.NO.3 | 5′-CAGCAGAGAGGCGAAATGT-3′ |
| SEQ.ID.NO.4 | 5′-AGT TTG TTT ATC TCA AGG C-3′ |
| SEQ.ID.NO.5 | 5′-AAT TAT TGA TTC TGT AGG T-3′ |
| SEQ.ID.NO.6 | 5′-GCA TGG AAG TCT CCA TGA G-3′ |
| SEQ.ID.NO.7 | 5′-ACT CAA ATT GGG GTC ACA G-3′ |
| SEQ.ID.NO.8 | 5′-TAC AAA GAA GTA GGT CCT A-3′ |
| SEQ.ID.NO.9 | 5′-GAT TTG GCA AGC GTT GGT T-3′ |
| SEQ.ID.NO.10 | 5′-ATG CTC TTG GGA TAA TTT G-3′ |
| SEQ.ID.NO.11 | 5′-CCA AGA AGT GCT GCA TTT C-3′ |
| SEQ.ID.NO.12 | 5′-CAC TGG TCT TTG GTC TCT C-3′ |
| SEQ.ID.NO.13 | 5′-AGA AGA ACC TGT CTT TGA A-3′ |
| SEQ.ID.NO.14 | 5′-CAG GGT CCA TCA TCT GTC T-3′ |

Antisense sequences SEQ.ID.NO.3-6 exhibited approximately 75-85% inhibition of MMP-12 mRNA levels relative to control. Antisense sequences SEQ.ID.NO.7-10 exhibited approximately 65-75% inhibition of MMP-12 mRNA levels, relative to control. Antisense sequences SEQ.ID.NO.11-14- exhibited approximately 50-65% inhibition of MMP-12 mRNA levels, relative to control.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

### REFERENCES

Ausubel, F. M. et al.. Current Protocols in Molecular Biology, Volume 2, pp.11.2.1-11.2.22, John Wiley & Sons, Inc., 1991.

Ausubel F M, Brent R, Struhl K, Smith J A. Short Protocols in Molecular Biology: A Compendium of Methods from Current Protocols in Molecular Biology, John Wiley & Sons; 2nd edition, April 1992.

Ausubel, F. M. et al.. Current Protocols in Molecular Biology, Volume 1, pp. 4.1.1-4.2.9 and 4.5.1-4.5.3, John Wiley & Sons, Inc., 1993.

Ausubel, F. M. et al.. Current Protocols in Molecular Biology, Volume 2, pp. 11.12.1-11.12.9, John Wiley & Sons, Inc., 1997.

Ausubel, F. M. et al.. Current Protocols in Molecular Biology, Volume 2, pp. 10.16.1-10.16.11, John Wiley & Sons, Inc., 1998.

Axelsson L-G, Landstrom E, Bylund-Fellenius A-C. Experimental colitis induced by dextran sulphate sodium in mice: beneficial effects of sulfasalazine and olsalazine. Aliment Pharmacol Therap 1998;12:925-934.

Church G M, Gilbert W (1984) Genomic sequencing. Proc Natl Acad Sci U S A. ;81:1991-5.

Cooper H S, Murthy S N, Shah R S, Sedergran D J. Clinicopathologic study of dextran sulfate sodium experimental murine colitis. Lab Invest 1993 August;69(2):238-49.

Docherty A J, Murphy G. The tissue metalloproteinase family and the inhibitor TIMP: a study using cDNAs and recombinant proteins. Ann Rheum Dis 1990 Jun;49(1):469-79.

Emonard H, Grimaud JA. Matrix metalloproteinases. A review. Cell Mol Biol 1990;36(2):131-53.

Franz A H, Liu R, Song A, Lam K S, Lebrilla C B.High-throughput one-bead-one-compound approach to peptide-encoded combinatorial libraries: MALDI-MS analysis of single TentaGel beads. J Comb Chem. 2003 March-April;5(2):125-37

Gearing A J, Beckett P, Christodoulou M, Churchill M, Clements J, Davidson A H, Drummond A H, Galloway W A, Gilbert R, Gordon J L, et al.. Processing of tumour necrosis factor-alpha precursor by metalloproteinases. Nature Aug. 18, 1994;370(6490):555-7.

Hautamaki R D, Kobayashi D K, Senior R M, Shapiro S D. Requirement for macrophage elastase for cigarette smoke-induced emphysema in mice. Science Sep. 26, 1997;277(5334):2002-4.

Hooper N M. Families of zinc metalloproteases. FEBS Lett Oct. 31, 1994;354(1):1-6.

Idson. in Pharmaceutical Dosage Forms, Lieberman, Rieger and Banker (Eds.), 1988, Marcel Dekker, Inc., New York, N.Y., volume 1, p. 199.

Jensen S, Gassama MP, Heidmann T. Taming of transposable elements by homologydependent gene silencing. Nat Genet 1999 February;21(2):209-12.

Ketting RF, Haverkamp TH, van Luenen HG, Plasterk RH. Mut-7 of C. elegans, required for transposon silencing and RNA interference, is a homolog of Werner syndrome helicase and RNaseD. Cell Oct. 15, 1999;99(2):133.41.

Lee VH, Yamamoto A, Kompella UB. Mucosal penetration enhancers for facilitation of peptide and protein drug absorption. Crit Rev Ther Drug Carrier Syst 1991;8(2):91-192.

Lucy AP, Guo HS, Li WX, Ding SW. Suppression of post-transcriptional gene silencing by a plant viral protein localized in the nucleus. EMBO J Apr. 3, 2000; 19(7): 1672-80.

Maniatis T, Sambrook J, Fritsch EF. Molecular Cloning, A laboratory Manual, Appendix A.1 Cold Spring Habor Laboratory; ₂nd Ed (1989).

Matrisian LM. The matrix-degrading metalloproteinases. Bioessays 1992 July;14(7):455-63.

McGeehan GM, Becherer JD, Bast RC Jr, Boyer CM, Champion B, Connolly KM, Conway JG, Furdon P, Karp S, Kidao S, et al.. Regulation of tumour necrosis factor-alpha processing by a metalloproteinase inhibitor. Nature Aug. 18, 1994;370(6490):558-61.

Mohler K M, Sleath P R, Fitzner J N, Cerretti D P, Alderson M, Kerwar S S, Torrance D S, Otten-Evans C, Greenstreet T, Weerawarna K, et al.. Protection against a lethal dose of endotoxin by an inhibitor of tumour necrosis factor processing. Nature Jul. 21, 1994;370(6486):218-20.

Morris D G, Huang X, Kaminski N, Wang Y, Shapiro S D, Dolganov G, Glick A, Sheppard D. Loss of integrin alpha v beta 6-mediated TGF-beta activation causes Mmpl2-dependent emphysema. Nature Mar. 13, 2003;422(6928):169-73.

Okayasu I, Hatakeyama S, Yamada M, Ohkusa T, Inagaki Y, Nakaya R A. Novel method in the induction of reliable experimental acute and chronic ulcerative colitis in mice. Gastroenterology 1990 March;98(3):694-702.

Rasmussen H S, McCann P P. Matrix metalloproteinase inhibition as a novel anticancer strategy: a review with special focus on batimastat and marimastat. Pharmacol Ther 1997;75(l):69-75.

Ratcliff F G, MacFarlane S A, Baulcombe D C. Gene silencing without DNA. ma-mediated cross-protection between viruses Plant Cell 1999 July;11(7):1207-16.

Rosoff, in Pharmaceutical Dosage Forms, Lieberman, Rieger and Banker (Eds.). 1988, Marcel Dekker, Inc., New York, N.Y., volume 1, p. 245.

Shapiro S D, Griffin G L, Gilbert D J, Jenkins N A, Copeland N G, Welgus H G, Senior R M, Ley T J. Molecular cloning, chromosomal localization, and bacterial expression of a murine macrophage metalloelastase. J Biol Chem Mar 5, 1992;267(7):4664-71.

Shapiro S D, Kobayashi D K, Ley T J. Cloning and characterization of a unique elastolytic metalloproteinase produced by human alveolar macrophages. J Biol Chem November 15;268(32):23824-9.

Shipley J M, Wesselschmidt R L, Kobayashi D K, Ley T J, Shapiro S D. Metalloelastase is required for macrophage-mediated proteolysis and matrix invasion in mice. Proc Natl Acad Sci U S A Apr. 30, 1996;93(9):3942-6.

Tabara H, Sarkissian M, Kelly W G, Fleenor J, Grishok A, Timmons L, Fire A, Mello CC. The rde-1 gene, RNA interference, and transposon silencing in C. elegans. Cell Oct 15, 1999;99(2): 123-32.

von Stein OD. Isolation of differentially expressed genes through subtractive suppression hybridization. Methods Mol Biol 2001; 175:263-78.

1. A compound 8 to 50 nucleobases in length targeted to a nucleic acid molecule encoding metalloproteinase 12 (MMP-12), characterized in that said compound specifically hybridises to and inhibits the translation of MMP-12 protein.

2. A compound according to claim 1, wherein the target sequence is SEQ ID NO. 1 or equivalent functional homologues thereof.

3. A compound according to claim 1, wherein the target sequence is SEQ ID NO. 2 or equivalent functional homologues thereof.

4. The compound according to claim 1, wherein said compound is an antisense oligonucleotide complementary to the mRNA.

5. The compound according to claim 1, wherein the oligonucleotide is a DNA molecule.

6. The compound according to claim 1, wherein the oligonucleotide is a RNA molecule.

7. The compound according to claim 4, wherein the antisense oligonucleotide has a sequence selected from the group consisting of SEQ ID NO. 3-14.

8. The compound according to claim 4, wherein the oligonucleotide is RNAi comprising at least an 8 nucleotide portion of a sequence selected from the group of SEQ.ID.NO 3- 14. and having a total length of no more than 25 nucleotides.

9. A compound 8 to 50 nucleobases in length targeted to a nucleic acid molecule encoding MMP-12, said compound being an antisense oligonucleotide which specifically hybridizes to and inhibits the translation of MMP-12 in a mammal, characterized in that the compound is chemically modified by substitution in a non-bridging oxygen atom of the antisense nucleic acid backbone with a moiety selected from the group consisting of methane phosphate, methyl phosphate, and phosphorothioate.

10. The compound according to claim 9, wherein the substitution occurs at one or more nucleotides selected from the 3′ end or the 5 ′ end or both.

11. The compound according to claim 9, wherein the substitution occurs at one or more nucleotides at any position along the entire length of said oligonucleotide.

12. The compound according to any one of the preceding claims, wherein said compound is an antisense oligonucleotide composed of DNA or RNA or an analogue or mimic of DNA or RNA including but not restricted to the following: methylphosphonate, N3′->P5′-phosphoramidate, morpholino, peptide nucleic acid (PNA), locked nucleic acid (LNA), arabinosyl nucleic acid (ANA), fluoro-arabinosyl nucleic acid (FANA) methoxyethyl nucleic acid (MOE).

13. The compound according to claim 1, wherein said compound is an antisense oligonucleotide that is a homo or heteropolymer containing combinations of the above DNA or RNA or analogues or mimics of DNA or RNA.

14. The compound according to any one of claims 2 -8, wherein in that said oligonucleotide comprises at least one modified sugar moiety nucleobase.

15. The compound of claim 14, wherein the modified sugar moiety is a 2′-O-methoxyethyl sugar moiety.

16. A composition comprising the compound according to any one of the preceding claims and a pharmaceutically acceptable carrier or diluent.

17. The composition of claim 16, wherein said composition further comprises a colloidal dispersion system.

18. A method of inhibiting the translation of MMP-12 in cells or tissues, wherein said cells or tissues are contacted with the compound of any one of claims 1 -15 thereby inhibiting the translation of MMP-12.

19. A method of inhibiting the translation of MMP-12 in cells or tissues, wherein said cells or tissues are contacted with the composition of any one of claims 16 -17 thereby inhibiting the translation of MMP-12.

20. The method according to claim 18 or 19, wherein the inhibition of the MMP-12 expression suppresses a MMP-12 dependent process in a human subject.

21. The method according to claim 20, wherein the MMP-12 dependent process is one of inflammatory bowel disease, such as ulcerative colitis and Crohn's disease, rheumatoid arthritis, psoriasis, emphysema and asthma.

22. A method of preventing, alleviating or treating a MMP-12 dependent disorder in a human patient, characterized in that MMP-12 expression is suppressed in one or more cells in said patient.

23. A method of preventing, alleviating or treating a MMP-12 dependent disorder in a human patient, characterized in that the level of MMP-12 is suppressed in one or more cells in said patient.

24. The method according to claim 22 or 23, wherein said MMP-12 dependent disorder is one of inflammatory bowel disease, such as ulcerative colitis and Crohn 's disease, rheumatoid arthritis, psoriasis, emphysema and asthma.

25. A recombinant nucleotide sequence comprising a compound according to any one of claim 1-15.

26. A recombinant expression vector comprising the recombinant nucleotide sequence according to claim 25.

27. The recombinant expression vector according to claim 26, wherein the vector is of eukaryotic or prokaryotic origin.

28. A method of inhibiting the expression of MMP-12 in cells or tissues, wherein said cells or tissues is contacted in vivo or in vitro with the recombinant nucleotide sequence expressed by the recombinant vector according to claim 27.

29. A recombinant host cell produced by the method of claim 28.

30. A transgenic non-human animal, wherein said animal carries at least one sequence according to claim 7 functionally inserted in at least one cell.

31. The transgenic animal according to claim 30, wherein the at least one functionally inserted sequence is over-expressed.

32. A method of inhibiting the expression of MMP-12 in cells or tissues, wherein a composition according to claim 16 or 17 is administered to a human in a therapeutically effective dose together with a pharmaceutically acceptable carrier.

33. A method of diagnosing inflammatory bowel disease in a human subject, wherein the method comprises screening for the presence or absence of the expression of MMP12 and the expression of MMP-12 is an indication of inflammatory bowel disease.


SEQUENCE LISTING (23 sequences)

SEQ ID NO: 1 - DNA, 1778 bp, Homo sapiens
  gene (1)..(1778): SEQ ID NO.1; cDNA human MMP-12, Genebank acc. no. NM-002426
tagaagtttacaatgaagtttcttctaatactgctcctgcaggccactgcttctggagctcttcccctgaacagctctacaagcctggaaaaaaataatgtgctatttggtgagagatacttagaaaaattttatggccttgagataaacaaacttccagtgacaaaaatgaaatatagtggaaacttaatgaaggaaaaaatccaagaaatgcagcacttcttgggtctgaaagtgaccgggcaactggacacatctaccctggagatgatgcacgcacctcgatgtggagtccccgatctccatcatttcagggaaatgccaggggggcccgtatggaggaaacattatatcacctacagaatcaataattacacacctgacatgaaccgtgaggatgttgactacgcaatccggaaagctttccaagtatggagtaatgttacccccttgaaattcagcaagattaacacaggcatggctgacattttggtggtttttgcccgtggagctcatggagacttccatgcttttgatggcaaaggtggaatcctagcccatgcttttggacctggatctggcattggaggggatgcacatttcgatgaggacgaattctggactacacattcaggaggcacaaacttgttcctcactgctgttcacgagattggccattccttaggtcttggccattctagtgatccaaaggctgtaatgttccccacctacaaatatgtcgacatcaacacatttcgcctctctgctgatgacatacgtggcattcagtccctgtatggagacccaaaagagaaccaacgcttgccaaatcctgacaattcagaaccagctctctgtgaccccaatttgagttttgatgctgtcactaccgtgggaaataagatctttttcttcaaagacaggttcttctggctgaaggtttctgagagaccaaagaccagtgttaatttaatttcttccttatggccaaccttgccatctggcattgaagctgcttatgaaattgaagccagaaatcaagtttttctttttaaagatgacaaatactggttaattagcaatttaagaccagagccaaattatcccaagagcatacattcttttggttttcctaactttgtgaaaaaaattgatgcagctgtttttaacccacgtttttataggacctacttctttgtagataaccagtattggaggtatgatgaaaggagacagatgatggaccctggttatcccaaactgattaccaagaacttccaaggaatcgggcctaaaattgatgcagtcttctattctaaaaacaaatactactatttcttccaaggatctaaccaatttgaatatgacttcctactccaacgtatcaccaaaacactgaaaagcaatagctggtttggttgttagaaatggtgtaattaatggtttttgttagttcacttcagcttaataagtatttattgcatatttgctatgtcctcagtgtaccactacttagagatatgtatcataaaaataaaatctgtaaaccataggtaatgattatataaaatacataatatttttcaattttgaaaactctaattgtccattcttgcttgactctactattaagtttgaaaatagttaccttcaaagcaagataattctatttgaagcatgctctgtaagttgcttcctaacatccttggactgagaaattatacttacttctggcataactaaaattaagtatatatattttggctcaaataaaattg

SEQ ID NO: 2 - DNA, 1790 bp, Murinae gen. sp.
  gene (1)..(1790): SEQ ID NO.2; cDNA murine MMP-12, Genebank acc. no. M82831
atgaaatttctcatgatgattgtgttcttacaggtatctgcctgtggggctgctcccatgaatgacagtgaatttgctgaatggtacttgtcaagattttatgattatggaaaggacagaattccaatgacaaaaacaaaaaccaatagaaacttcctaaaagaaaaactccaggaaatgcagcagttctttgggctagaagcaactgggcaactggacaactcaactctggcaataatgcacatccctcgatgtggagtgcccgatgtacagcatcttagagcagtgccccagaggtcaagatggatgaagcggtacctcacttacaggatctataattacactccggacatgaagcgtgaggatgtagactacatatttcagaaagctttccaagtctggagtgatgtgactcctctaagattcagaaagcttcataaagatgaggctgacattatgatactttttgcatttggagctcacggagacttcaactattttgatggcaaaggtggtacactagcccatgttttttatcctggacctggtattcaaggagatgcacattttgatgaggcagaaacgtggactaaaagttttcaaggcacaaacctcttccttgttgctgttcatgaacttggccattccttggggctgcagcattccaataatccaaagtcaataatgtaccccacctacagataccttaaccccagcacatttcgcctctctgctgatgacatacgtaacattcagtccctctatggagccccagtgaaacccccatccttgacaaaacctagcagtccaccatcaactttctgtcaccaaagcttgagttttgatgctgtcacaacagtgggagagaaaatccttttctttaaagactggttcttctggtggaagcttcctgggagtccagccaccaacattacttctatttcttccatatggccaagcatcccatctgctattcaagctgcttacgaaattgaaagcagaaatcaacttttcctttttaaagatgagaagtactggttaataaacaacttagtaccagagccacactatcccaggagcatatattccctgggcttctctgcatctgtgaagaaggttgatgcagctgtctttgacccacttcgccaaaaggtttatttctttgtggataaacactactggaggtatgatgtgaggcaggagctcatggaccctgcttaccccaagctgatttccacacacttcccaggaatcaagcctaaaattgatgcagtcctctatttcaaaagacactactacatcttccaaggagcctatcaattggaatatgaccccctgttccgtcgtgtcaccaaaacattgaaaagtacaagctggtttggttgttaggaagaatgtagtgaagggtgcttgctggtttttcagttttataagtatatttattacatattcactctatgctcagggtgtaactatgtggcaataatgtaacaggaaataaggggaggtgtacaggtcacacacacatagttacacagaaaagtgcttttacaaaattaacctcttttaggaacttttttcacttcattctattcttaattttgaaagtgcatggttcagaggccaactggtttatctgtaagttgttttctaacaaccttcaagtagaatattagaattagaattactctcttgtctttactgaaatgtaacatgttttgttttctttaaataattgaaagaaagtgaaaaaaaaaaaaaaaaaaaaaaaaaaacggaattcccgggga

SEQ ID NO: 3 - DNA, 19 bp, Artificial
  misc_feature (1)..(19): SEQ ID NO.3, antisense oligonucleotide
cagcagagaggcgaaatgt

SEQ ID NO: 4 - DNA, 19 bp, Artificial
  misc_feature (1)..(19): SEQ ID NO.4, antisense oligonucleotide
agtttgtttatctcaaggc

SEQ ID NO: 5 - DNA, 19 bp, Artificial
  misc_feature (1)..(19): SEQ ID NO. 5, antisense oligonucleotide
aattattgattctgtaggt

SEQ ID NO: 6 - DNA, 19 bp, Artificial
  misc_feature (1)..(19): SEQ ID NO. 6, antisense oligonucleotide
gcatggaagtctccatgag

SEQ ID NO: 7 - DNA, 19 bp, Artificial
  misc_feature (1)..(19): SEQ ID NO. 7, antisense oligonucleotide
actcaaattggggtcacag

SEQ ID NO: 8 - DNA, 19 bp, Artificial
  misc_feature (1)..(19): SEQ ID NO. 8, antisense oligonucleotide
tacaaagaagtaggtccta

SEQ ID NO: 9 - DNA, 19 bp, Artificial
  misc_feature (1)..(19): SEQ ID NO. 9, antisense oligonucleotide
gatttggcaagcgttggtt

SEQ ID NO: 10 - DNA, 19 bp, Artificial
  misc_feature (1)..(19): SEQ ID NO. 10, antisense oligonucleotide
atgctcttgggataatttg

SEQ ID NO: 11 - DNA, 19 bp, Artificial
  misc_feature (1)..(19): SEQ ID NO. 11, antisense oligonucleotide
ccaagaagtgctgcatttc

SEQ ID NO: 12 - DNA, 19 bp, Artificial
  misc_feature (1)..(19): SEQ ID NO. 12, antisense oligonucleotide
cactggtctttggtctctc

SEQ ID NO: 13 - DNA, 19 bp, Artificial
  misc_feature (1)..(19): SEQ ID NO. 13, antisense oligonucleotide
agaagaacctgtctttgaa

SEQ ID NO: 14 - DNA, 19 bp, Artificial
  misc_feature (1)..(19): SEQ ID NO. 14, antisense oligonucleotide
cagggtccatcatctgtct

SEQ ID NO: 15 - DNA, 60 bp, Artificial
  misc_feature (1)..(60): SEQ ID NO. 15; Oligo-dT-primer dT-joint
tagtctatgatcgtcgacggctgatgaagcggccgctggagttttttttttttttttttv

SEQ ID NO: 16 - DNA, 18 bp, Artificial
  misc_feature (1)..(19): SEQ ID NO. 16; joint-Not primer
tgatgaagcggccgctgg

SEQ ID NO: 17 - DNA, 20 bp, Artificial
  misc_feature (1)..(20): SEQ ID NO. 17; jonit-Sal primer
ttcatcagccgtcgacgatc

SEQ ID NO: 18 - DNA, 23 bp, Artificial
  misc_feature (1)..(23): SEQ ID NO. 18; Sport-Not primer
cgtaagcttggatcctctagagc

SEQ ID NO: 19 - DNA, 23 bp, Artificial
  misc_feature (1)..(23): SEQ ID NO. 19; Sport-Sal primer
tgcaggtaccggtccggaattcc

SEQ ID NO: 20 - DNA, 24 bp, Artificial
  misc_feature (1)..(24): SEQ ID NO. 20; MMP-12 forward primer
gacttcctactccaacgtatcacc

SEQ ID NO: 21 - DNA, 24 bp, Artificial
  misc_feature (1)..(24): SEQ ID NO. 21; MMP-12 reverse primer
ctcagtccaaggatgttaggaagc

SEQ ID NO: 22 - DNA, 24 bp, Artificial
  misc_feature (1)..(24): SEQ ID NO. 22; alpha-actin forward primer
gtgcagggtattaacgtgtcaggg

SEQ ID NO: 23 - DNA, 28 bp, Artificial
  misc_feature (1)..(28): SEQ ID NO. 23; alpha-actin reverse primer
ccaactcaaagcaagtaacagcccacgg